(19) United States
(12) Patent Application Publication
(10) Pub. No.: US 2025/0352584 A1
(43) Pub. Date: Nov. 20, 2025

(54) CELL DELIVERY VEHICLE AND METHODS OF USING THE SAME

(71) Applicant: BLUEROCK THERAPEUTICS LP, Cambridge, MA (US)

(72) Inventors: Ema Cristina OLAH, Cambridge, MA (US); Virginie Florence Margherita COINDRE, Cambridge, MA (US); Ali AFFAR, Cambridge, MA (US); Genevieve Ann CONANT, Cambridge, MA (US); Shantanu Vijay LALE, Cambridge, MA (US); Xinpei LI, Cambridge, MA (US); Tanvia Mahesh SHAH, Cambridge, MA (US)

(73) Assignee: BLUEROCK THERAPEUTICS LP, Cambridge, MA (US)

(21) Appl. No.: 18/871,376

(22) PCT Filed: May 12, 2023
(86) PCT No.: PCT/US2023/021961

§ 371 Date: Dec. 3, 2024

Related U.S. Application Data
(60) Provisional application No. 63/348,709, filed on Jun. 3, 2022.

(51) Int. Cl. A61K 35/30; A61K 9/00; A61K 47/02; A61K 47/20; A61K 47/26; A61K 47/34; A61K 47/36; A61K 47/42

(52) U.S. Cl. CPC A61K 35/30; A61K 9/0019; A61K 47/02; A61K 47/20; A61K 47/26; A61K 47/34; A61K 47/36; A61K 47/42

(57) ABSTRACT

The present disclosure is directed to various compositions that include one or more energy source components; one or more pH buffers; one or more salts; and one or more stabilizing agents and one or more cell types.

### BACKGROUND OF THE DISCLOSURE

Various biological cells and/or tissues are stored at freezing temperatures, for varying amounts of time and in various materials, such as cryoprotectants. These cryoprotectants used during storage are to be removed or substantially removed from the biological cells and/or tissues after or during the thawing process, so that those biological cells and/or tissues can be utilized.

Typically, a container containing the biological cells and/or tissues is removed from freezing temperature storage, thawed, the content of the container are “washed” or diluted with a cell wash solution to remove some, a majority or all of the cryoprotectant. Typical cell “wash” solutions have many, many components, including but not limited to: one or more amino acids such as Glycine, L-Alanine, L-Arginine hydrochloride, L-Asparagine-H₂O, L-Glutamine, L-Cysteine, L-Histidine hydrochloride-H₂O, L-Isoleucine, L-Leucine, L-Lysine hydrochloride, L-Methionine, L-Phenylalanine, L-Proline, L-Serine, L-Threonine, L-Tryptophan, L-Tyrosine, and L-Valine; one or more glycolysis components, such as Dextrose and Sodium Pyruvate; one or more pH controlling components and/or pH buffers such as 4-(2-hydroxyethyl)-1-piperazineethanesulfonic acid (HEPES) and Phenol Red; one or more proteins, for example albumins such as Human Serum Albumin; one or more vitamins such as Ascorbic Acid, Choline Chloride, D-Calcium pantothenate, Folic Acid, Niacinamide, Pyridoxal hydrochloride, Riboflavin, Thiamine hydrochloride, Vitamin B12 and i-Inositol; and one or more salts such as Calcium Chloride (CaCl₂)), Ferric Nitrate (Fe(NO₃)3″9H₂O), Magnesium Chloride (MgCl), Potassium Chloride (KCl), Sodium Bicarbonate (NaHCO₃), Sodium Chloride (NaCl), Sodium Phosphate monobasic (NaH₂PO₄—H₂O), and Zinc sulfate (ZnSO₄-7H₂O).

After dilution with the cell wash solution, the container can undergo one or more settling procedures, such as centrifugation or settling over time. The supernatant can then be wholly or partially removed from the container, so that all or a majority of the cells remain. The process can then be repeated with addition of cell wash solution again to the container followed by a settling procedure one or more times.

After the cells are “washed” the remaining biological cells and/or tissues, which can be in the same storage container or could have been transferred to a different container, are resuspended with a cell delivery solution (transplantation media) so that the biological cells and/or tissues can be used for whatever procedure that is needed. Typically, this cell delivery solution has some similarities in composition with the cell wash solution, with the cell delivery solution being the same or different concentration of components as compared to the cell wash solution or having additional components.

What is desired is a cell wash solution and/or a cell delivery solution that has an appropriate shelf-life at standard storage temperatures, has few or no components of human or mammalian origin, a substantially stable pH value over days, weeks or longer, a substantially physiological osmolarity, and that maintains a better cell health as commercially available solutions. In addition, the cell delivery solution composition can be designed to sufficiently prevent cell settling, to allow for an accurate dose assurance during preparation and administration. This accurate dose assurance and delivery can be the result of the cell delivery solution's ability to maintain cells substantially in suspension through 8 hours or longer.

Embodiments of the present disclosure provide solutions and methods that address the above needs.

### SUMMARY OF THE DISCLOSURE

The present disclosure is directed to various formulations or compositions that include one or more energy source components; one or more pH buffers; one or more salts; and one or more stabilizing agents. The present disclosure is also directed to a cell wash solution (CWS), a cell delivery solution (CDS) and cell suspension for injection (CSI). The cell suspension for injection provides cells in suspension for several hours, while maintaining a sufficient level of cell health.

### BRIEF DESCRIPTION OF THE DRAWINGS

The present disclosure will be better understood by reference to the following drawings, which are provided as illustrative of certain embodiments of the subject application, and not meant to limit the scope of the present disclosure.

FIG. 1 is a graphical illustration of cell viability over time.

FIG. 2 is a graphical illustration of the percentage of apoptotic cells over time.

FIG. 3 are graphical illustrations of cell health qualities over time.

FIG. 4 is a graphical illustration of cell dose over time.

FIG. 5A-5C are graphical illustrations of cell viability and dose over time.

FIG. 6 is a graphical illustration of the percentage of apoptotic cells over time.

FIG. 7 are photographs of cells cultured after 8 hours of storage.

FIG. 8 is a table of photographs of cells in suspension or not when stored in different media.

FIG. 9 is a graphical illustration of cell settling over time, using a scoring system.

FIG. 10 is a visual representation of a cell settling scoring system.

FIGS. 11A-11B are graphical illustrations of the variability in dose sampling over time.

FIG. 12 is a graphical illustration of the potency of the cells over time.

FIGS. 13A and 13B are graphical illustrations of live cell concentrations for cells stored over time in different solutions.

FIG. 14A is a graphical illustration of cell viability for cells stored over time in different solutions.

FIG. 14B is a graphical illustration of total cell concentration for cells stored over time in different solutions.

FIG. 15 is a number of representative images of cells stored over time in different solutions.

### DETAILED DESCRIPTION OF THE DISCLOSURE

In the discussion and claims herein, the term “about” indicates that the value listed may be somewhat altered, as long as the alteration does not result in nonconformance of the process. For example, for some elements the term “about” can refer to a variation of +0.1%, for other elements, the term “about” can refer to a variation of +1% or +10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either completely flat, or so nearly flat that the effect would be the same as if it were completely flat.

As used herein terms such as “a”, “an” and “the” are not intended to refer to only a singular entity but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element, and a second element, are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the term “stabilizing agent” refers to any component that can act to reduce or prevent degradation of other solution components.

As used herein, the term “energy source” refers to any component that can provide chemical energy to one or more cells.

As used herein, the term “pH indicator” refers to any component or substance that changes its properties in response to a change in pH. Such changes in properties include a change in optical properties, such as a color change.

As used herein the term “vitamin” includes any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) either obtained naturally from plant and animal foods or synthetically made, as well as pro-vitamins, derivatives, and/or analogs thereof.

As used herein, the term “cells” can refer to any one or more types of cell noted in this or any other paragraph of the disclosure, or substitutes and/or equivalents thereof. In some embodiments, the cells are neural cells. The cells can be Embryonic Stem Cell-derived or induced-Pluripotent Stem Cell-derived. In some embodiments, the cells are dopaminergic neuron cells, engraftable midbrain dopaminergic neurons, midbrain dopaminergic neurons, authentic midbrain dopamine (DA) neurons, midbrain dopaminergic neuron progenitor cells, dopaminergic neuron progenitor cells, dopaminergic neuron precursor cells, and, in some embodiments, may be iPSC-derived dopaminergic neuron cells.

In some embodiments, “cells” can refer to floor plate midbrain progenitor cell floor-plate derived DA neurons. As used herein, the term “floor-plate derived DA neurons” or “authentic midbrain DA neurons” or “midbrain fate FOXA2+LMX1A+dopamine (DA) neurons” or “floor plate midbrain dopamine (DA) neuron” or “engraftable midbrain DA neuron” or “mDA neuron” or “FOXA2+LMX1A+TH+” or “FOXA2/LMX1A/TH” or “FOXA2+LMX1A+NURR1+TH+” or “FOXA2/LMX1A/NURR1/TH” refers to an engraftable midbrain DA neuron population obtained in any suitable way.

As used herein, cells used for obtaining floor plate midbrain progenitor cells and midbrain fate FOXA2/LMX1A+dopamine (DA) neurons are obtained from a variety of sources including embryonic and nonembryonic sources, for example, hESCs and nonembryonic hiPSCs, somatic stem cells, disease stem cells, i.e. isolated pluripotent cells and engineered derived stem cells isolated from Parkinson disease patients, cancer stem cells, human or mammalian pluripotent cells, etc. These cells for obtaining floor plate midbrain progenitor cells and midbrain fate FOXA2/LMX1A+dopamine (DA) neurons, or differentiated midbrain dopaminergic neuronal cells, “DMD” cells, can also be “cells” of the present disclosure.

As used herein, the term “stem cell” refers to a cell with the ability to divide for indefinite periods in culture and to give rise to specialized cells. A stem cell may be obtained from animals and patients, including humans; for example, a human stem cell refers to a stem cell that is human. A stem cell may be obtained from a variety of sources including embryonic and nonembryonic, such as umbilical cord cells, cells from children and cells from adults. For the purposes of the present inventions, adult stem cells in general refer to cells that were not originally obtained from a fetus, in other words, cells from babies, cast off umbilical cords, cast off placental cells, cells from children, cells from adults, etc.

As used herein, the term “umbilical cord blood stem cells” refer to stem cells collected from an umbilical cord at birth that have the capability to at least produce all of the blood cells in the body (hematopoietic).

As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin. The term “somatic cells” as used herein, refers to any cell of the body except sperm and egg cells. Somatic cells are diploid, meaning that they contain two sets of chromosomes, one inherited from each parent. Somatic cells include brain cells. Brain cells include, but are not limited to neurons, oligodendrocytes, astrocytes, microglia, perivascular macrophages, meningial macrophages, endothelial cells, pericytes, ependymal cells and blood cells.

The term “allogeneic cells” or “allogeneic stem cells” as used herein, refers to cells that are obtained from individuals belonging to the same species but are genetically dissimilar. The allogeneic stem cells of the disclosure are from a person other than the patient, either a matched related or unrelated donor.

As used herein, the term “neural lineage cell” refers to a cell that contributes to the nervous system (both central and peripheral) or neural crest cell fates during development or in the adult. The nervous system includes the brain, spinal cord, and peripheral nervous system. Neural crest cell fates include cranial, trunk, vagal, sacral, and cardiac, giving rise to mesectoderm, cranial cartilage, cranial bone, thymus, teeth, melanocytes, iris pigment cells, cranial ganglia, dorsal root ganglia, sympathetic/parasympathetic ganglia, endocrine cells, enteric nervous system, and portions of the heart.

As used herein, the term “adult stem cell” refers to a somatic stem cell, for one example, a “hematopoietic stem cell” which refers to a stem cell in babies, children and adults, that gives rise to all red and white blood cells and platelets.

As used herein, the term “embryonic stem cell” refers to a primitive (undifferentiated) cell that is derived from one of several sources, including but not limited to a preimplantation-stage embryo, an artificially created embryo, i.e. by in vitro fertilization, etc., capable of dividing without differentiating for a prolonged period in culture, and are known to have the capability to develop into cells and or tissues of the three primary germ layers, the ectoderm, the mesoderm, and the endoderm.

As used herein, the term “endoderm” refers to a layer of the cells derived from the inner cell mass of the blastocyst; it has the capability to give rise to lungs, other respiratory structures, and digestive organs, or generally “the gut” “in vivo” and a variety of cell types in vitro.

As used herein, the term “embryonic stem cell line” refers to a population of embryonic stem cells that have been cultured under in vitro conditions that allow proliferation without differentiation for up to days, months to years, for example, cells in a human WA-09 cell line.

As used herein, the term “human embryonic stem cell” or “hESC” refers to a type of pluripotent stem cells derived from early-stage human embryos, up to and including the blastocyst stage, that is capable of dividing without differentiating for a prolonged period in culture and are known to develop into cells and tissues of the three primary germ layers, the ectoderm, the mesoderm, and the endoderm.

The term “pluripotent stem cells” or “PSCs,” as used herein, has its usual meaning in the art, i.e., self-replicating cells that have the ability to develop into endoderm, ectoderm, and mesoderm cells. As used herein, PSCs include “genetically edited or modified PSCs”. In some embodiments PSCs are human PSCs. PSCs include embryonic stem cells (ESCs) and induced pluripotent stem cells (“iPS cells” or “iPSCs” or “hiPSCs”). The terms ES cells and iPS cells have their usual meaning in the art. As used herein “genetically edited or modified PSCs” include cells wherein an aEFla-IDUA overexpression cassette is engineered into the AAVSI safe harbor locus in human induced pluripotent stem cells (hiPSCs).

The term “non-pluripotent stem cells,” as used herein, has its usual meaning in the art, i.e. that the cell does not have the potential to differentiate into all of the three germ layers (i.e. endoderm, ectoderm, and mesoderm). Examples of such cells include umbilical cord blood stem cells and epidermal stem cells.

As used herein, the term “induced pluripotent stem cell” or “iPSC” refers to a type of pluripotent stem cell, similar to an embryonic stem cell, whereby somatic (adult) cells are reprogrammed to enter an embryonic stem cell-like state by being forced to express factors important for maintaining the “stemness” of embryonic stem cells (ESCs). Mouse iPSCs were reported in 2006 (Takahashi and Yamanaka), and human iPSCs were reported in late 2007 (Takahashi et al. and Yu et al.). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers. Unlike an embryonic stem cell an iPSC is formed artificially by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples of cell lines from induced cells, C14, C72, and the like. Another example of an iPSC is an adult human skin cell, or fibroblast cell, transformed with using genes (OCT4, SOX2, NANOG, LIN28, and KLF4) cloned into a plasmid for example, see, Yu, et al., Science DOI: 10.1126/science.1172482, herein incorporated by reference.

As used herein, the term “totipotent” refers to an ability to give rise to all cell types of the body plus all of the cell types that make up the extraembryonic tissues such as the placenta.

As used herein, the term “multipotent” refers to an ability to develop into more than one cell type of the body.

As used herein, the term “pluripotent” refers to a cell having the ability to give rise to at least two but often numerous different cell types of the body. Pluripotent cells often generate a teratoma after injection into an immunosuppressed mouse.

As used herein, the term “specialized cell” refers to a type of cell that performs a specific function in multicellular organisms. For example, groups of specialized cells, such as neurons, work together to form a system, such as a nervous system.

As used herein, the term “neuroectoderm” refers to a cell or cell fate found early in development or during pluripotent stem cell differentiation that can give rise to cells of the neural lineage.

It should be appreciated however, that the compositions can include other types of “cells” alone or in combination, such as mesenchymal stem cells, hematopoietic stem cells, embryonic stem cells or induced pluripotent stem cells, red blood cells, platelets, chondrocytes, skin cells, immune cells (e.g. tumor infiltrating lymphocytes, viral reconstitution T cells, dendritic cells, regulator T cells, macrophages), neural crest stem cells, neurons, glia, smooth muscle, cardiac tissue, chondrocytes, osteocytes, glial restricted progenitors, astrocytes, oligodendrocytes, neuroblast cells, megakaryoblasts, megakaryocytes, monoblasts, monocytes, macrophages, myeloid cells, myeloid dendritic cells, microglial cells, differentiated microglial cells, microglial progenitor cells, proerythroblasts, erythroblasts, normoblasts, reticulocytes, thrombocytes, myeloblasts, progranulocytes, neutrophilic myelocytes, neutrophilic band cells, neutrophils, eosinophilic myelocytes, eosinophilic band cells, eosinophils, basophilic myelocytes, basophilic band cells, basophils, committed lymphoid progenitors, pre-NK cells, NK lymphoblasts, NK cells, thymocytes, T-lymphoblasts, T-cells, plasmacytoid dendritic cells, pre-B cells, B-lymphoblasts, B cells, plasma cells, osteoblasts, chondrocytes, myoblasts, myotubes, fibroblasts, adipocytes, mesoderm, ectoderms, primordial germ cells, sperm, eggs, definitive endoderm, heart cells, or any other suitable type of cell. The heart cells can include, but are not limited to, cardiomyocytes, atrial cardiomyocytes, ventricular cardiomyocytes, fibroblasts (FBs), endothelial cells (ECs), pericytes, smooth muscle cells (SMCs), immune cells (myeloid and lymphoid), adipocytes, mesothelial cells and neuronal cells.

The term “microglial progenitor cells” as used herein, refer to PSC-derived myeloid cells.

The term “microglia” as used herein, refer to PSC-derived myeloid cells.

Additionally, “cells” can refer to myeloid cells enriched in microglial cells derived from unedited Human induced pluripotent stem cells (hiPSCs). The term “myeloid cells” as used herein, refer to PSC-derived cells and/or non-PSC-derived. PSC-derived cells are differentiated following a hemopoietic differentiation, express myeloid markers (including but not limited to: CD45, CD11b, CD33, CD14, CX3CR1), and are able to perform regular myeloid functions including but not limited to phagocytosis, respond to external stimuli, secretion of cytokines and polarization to pro-inflammatory or anti-inflammatory states. These myeloid cells will become the tissue-resident macrophages upon delivery to different organs in a live organism, e.g., they will become microglia when delivered into the brain of live animals, as they will express canonical microglia markers including but not limited to TMEM119, IBA1, CD163, CX3CR1, CD45, CD206. The payload of these cells can be replete with the complement of missing lysosomal enzymes and may not have any genetic modifications to include any additional payload.

In addition to any of the cells noted above as being “cells” of this disclosure, “cells” can refer to non-living biologics, including but not limited to endosomes and lipid-based vesicles. In further addition to any of the cells noted above as being “cells” of this disclosure, “cells” can refer to any cell and/or tissue disclosed in U.S. Pat. Nos. 10,280,398, 10,711,243 and International Application WO 2013/067362, the contents of each of which are incorporated herein in their entireties.

As used herein, the term “cell wash solution” or “CWS” is any solution that is added to a container containing cells and a cryoprotectant after removal of that container from a below 0° C. environment. As used herein, the term “cryoprotectant” refers to a substance that is used to reduce or eliminate cell damage caused by freezing and thawing processes which are inevitably accompanied by ice crystal formation and ionic and osmotic imbalance when cells and/or tissues are preserved at temperatures below 0° C. The cryoprotectant is not limited to a certain substance, as long as it is able to reduce cell damage during below 0° C. preservation. Examples thereof may include a permeating-type cryoprotectant such as dimethyl sulfoxide (DMSO), glycerol, propylene glycol, ethylene glycol, etc., or a non-permeating-type cryoprotectant such as sucrose, carboxymethylcellulose salts, carboxymethylcellulose (CMC), monosaccharide, disaccharide, etc., but are not limited thereto.

As used herein, the term “cell delivery solution” or “CDS” is any solution that is added to a container containing unwashed cells (directly on thawed cells) or washed cells so that the cells can be administered to a subject. The cell delivery solution can contain no, minimal or trace amounts of cryoprotectant and/or cell wash solution, which the cells were stored and/or washed in prior to contact with the cell delivery solution, that were not fully removed from the container after a supernatant discard process, which can occur after an optional centrifuge step of the container, which can optionally form a cell pellet or concentrated cell solution. This supernatant discard process reduces the concentration and/or removes components of a stored sample that are not desired for injection, such as the cryoprotectant. The cell delivery solution can be used to reconstitute the cell solution for injection (CSI) following thawing of cells prior to administration for clinical use.

As used herein, the term “cell suspension for injection” or “CSI” refers to either or both of the cell delivery solution (CDS) and/or the cell wash solution (CWS), in combination with one or more cells, with one or more optional, additional components. The cell suspension for injection can include the additional component of, for example, a cell wash buffer, such as, for example, compositions comprising phosphate buffer saline (PBS), compositions comprising 4-(2-hydroxyethyl)-1-piperazineethanesulfonic acid (HEPES), Bio-Plex Pro™ Cell Signaling Wash Buffer, and Cultrex™ 3-D Cell Wash Buffer. The cell suspension for injection may also contain trace amounts of cryoprotectant and/or cell wash solution that were not fully removed from the container after a supernatant discard process. However, the possible trace amounts of cryoprotectant and/or cell wash solution do not alter the efficacy of the cell suspension for injection.

The cell delivery solution and/or the cell wash solution can be used at a manufacturing site to create the CSI, which is the composition or product that is administered, e.g. injected into a subject. The manufacturing site can receive a plurality of cells, “wash” those cells, mix those cells with the cell delivery solution and/or the cell wash solution and then prepare those cells for administration, e.g. injection into a subject as a CSI. Injection type contemplated by the disclosure includes, but is not limited to intracerebroventricular (ICV), intravenous (IV), intramuscular (IM), intrathecal (IT). The manufacturing site can perform these steps in an aseptic environment and under appropriate environmental conditions. Additionally, the manufacturing site can perform quality control testing of the cells during any point of their preparation, prior to leaving the manufacturing site, to determine if the cells are sufficiently viable.

The present disclosure provides a “cell wash solution (CWS)” and a “cell delivery solution (CDS)”, either or both of which can be referred to as a “cell solution”. Both the cell wash solution and the cell delivery solution can be formulated to constitute various qualities, such as a (A) pH level of about 5.5 to about 9.0, or a pH level of about 6.0 to about 8.0, or a pH level of about 6.4 to about 7.8, or a pH level of about 6.8 to about 7.6, or a pH level of about 7.0 to about 7.5, or a pH level of about 7.2 to about 7.4, (B) (B) an osmolarity of about 100 to about 700 mOsm/L, or an osmolarity of about 150 to about 500 mOsm/L, or an osmolarity of about 200 to about 500 mOsm/L, or an osmolarity of about 225 to about 400 mOsm/L, or an osmolarity of about 250 to about 350 mOsm/L, or an osmolarity of about 270 to about 325 mOsm/L, or an osmolarity of about 280 to about 300 mOsm/L, and (C) a density of about 1.00 to about 1.30 g/mL, or a density of about 1.02 to about 1.20 g/mL, or a density of about 1.04 to about 1.15, or a density of about 1.05 to about 1.11 g/mL, g/mL, or a density of about 1.07 to about 1.09 g/mL, or a density of about 1.08 g/mL, a relatively low viscosity, and a good cell compatibility such that both the cell wash solution and the cell delivery solution are substantially not cytotoxic. Both the cell wash solution and the cell delivery solution are configured to have a sufficient shelf life at typical or standard storage conditions, making it ready to use for clinical applications.

These qualities can influence the ability of the cell delivery solution to maintain cells in suspension. As used herein, the term “suspension” refers to cells that are dispersed within a liquid. In this disclosure the cells dispersed within a liquid can remain dispersed for 0 to 104 hours or more. The shelf-life of the cells dispersed or suspended within a liquid in accordance with the present disclosure is up to about 104 hours. More specifically, in this disclosure the liquid can be the cell delivery solution that can maintain cells dispersed within it either without agitation (mixing) and/or homogenization or after agitation (mixing) and/or homogenization, for up to about 15 minutes, up to about 30 minutes, up to about 45 minutes, up to about 1 hour, up to about 90 minutes, up to about 2 hours, up to about 4 hours, up to about 6 hours, up to about 8 hours, up to about 12 hours, up to about 16 hours, up to about 20 hours, up to about 24 hours, up to about 30 hours, up to about 36 hours, up to about 42 hours, up to about 48 hours, up to about 56 hours, up to about 64 hours, up to about 72 hours, up to about 80 hours, up to about 88 hours, up to about 96 hours, up to about 104 hours, or more.

Both the cell wash solution and the cell delivery solution can include several components, including but not limited to one or more energy source components; one or more pH buffers; one or more salts; and one or more stabilizing agents. The several components of both the cell wash solution and the cell delivery solution can be combined in any suitable way to arrive at either the cell wash solution and the cell delivery solution, such as through the methods discussed in the Examples below. Both the cell wash solution and the cell delivery solution can be warmed or cooled to any appropriate temperature (e.g., about room temperature, or about 37° C., or about 4° C., or about 0° C., or about 2° C. to about 8° C., or about 1° C. to about 10° C., or about 0° C. to about 12° C.) before contact with any cells.

The one or more energy source components of either the cell wash solution or the cell delivery solution can comprise any suitable sugar, such as dextrose, fructose, galactose, glucose, lactose, maltose, sucrose. Either the cell wash solution or the cell delivery solution can comprise any amount of energy source(s) that achieve the desired effect, including but not limited to about 24.5 mM to about 24.8 mM, about 24.4 mM to about 24.9 mM, about 24.3 mM to about 25.0 mM, about 24.1 mM to about 25.2 mM, about 23.9 mM to about 25.4 mM, about 23.7 mM to about 25.6 mM, or about 23.5 mM, or about 23 mM, or about 22 mM, or about 21 mM, or about 20 mM or less, or about 26 mM, or about 27 mM, or about 28 mM, or about 29 mM, or about 30 mM or more.

The one or more pH buffers of either the cell wash solution or the cell delivery solution can comprise any suitable buffering agent, such as a zwitterionic organic chemical buffering agent, examples of which include, but are not limited to, 4-(2-hydroxyethyl)-1-piperazineethanesulfonic acid (HEPES), sodium bicarbonate, 4-Morpholinepropanesulfonic acid, 3-propanesulfonic acid (MOPS), and 2-(N-morpholino) ethanesulfonic acid (MES). Either the cell wash solution or the cell delivery solution can comprise any amount of pH buffer(s) that achieves the desired effect, including but not limited to about 10.6 mM to about 10.9 mM, about 10.5 mM to about 11.0 mM, about 10.4 mM to about 11.1 mM, about 10.2 mM to about 11.3 mM, about 10.0 mM to about 11.5 mM, about 9.8 mM to about 11.7 mM, or about 9.5 mM, or about 9 mM, or about 8 mM, or about 7 mM, or about 6 mM or less, or about 12 mM, or about 13 mM, or about 14 mM, or about 15 mM, or about 16 mM or more of pH buffer.

The one or more salts of either the cell wash solution or the cell delivery solution can comprise any suitable salt, such as one or more salts are selected from the group consisting of calcium chloride, magnesium chloride, potassium chloride, sodium phosphate monobasic and sodium chloride.

Either the cell wash solution or the cell delivery solution can comprise any amount of calcium chloride to achieve the desired effect, including but not limited to about 1.6 mM to about 1.9 mM, or about 1.5 mM to about 2.0 mM, or about 1.4 mM to about 2.1 mM, or about 1.3 mM to about 2.2 mM, or about 1.1 mM to about 2.4 mM, or about 0.9 mM to about 2.6 mM, or about 0.7 mM to about 2.8 mM, or about 0.5 mM, or about 0.3 mM, or about 0.1 mM or less, or about 3 mM, or about 3.5 mM, or about 4 mM, or about 5 mM or more.

Either the cell wash solution or the cell delivery solution can comprise any amount of magnesium chloride to achieve the desired effect, including but not limited to about 0.7 mM to about 1.0 mM, or about 0.6 mM to about 1.1 mM, or about 0.5 mM to about 1.2 mM, or about 0.4 mM to about 1.4 mM, or about 0.3 mM to about 1.6 mM, or about 0.2 mM to about 1.9 mM, or about 0.1 mM to about 2.2 mM, or about 0.05 mM or less, or about 2.5 mM, or about 3.0 mM or more.

Either the cell wash solution or the cell delivery solution can comprise any amount of potassium chloride to achieve the desired effect, including but not limited to about 5.1 mM to about 5.4 mM, or about 5.0 mM to about 5.5 mM, or about 4.9 mM to about 5.7 mM, or about 4.8 mM to about 5.8 mM, or about 4.6 mM to about 6.0 mM, or about 4.4. mM to about 6.2 mM, or about 4.2 mM to about 6.4 mM, or about 4.0 mM, or about 3.5 mM, or about 3.0 mM or less, or about 6.5 mM, or about 7.0 mM, or about 7.5 mM, or about 8.0 mM or more.

Either the cell wash solution or the cell delivery solution can comprise any amount of sodium phosphate monobasic to achieve the desired effect, including but not limited to about 0.88 mM to about 0.91 mM, or about 0.87 mM to about 0.92 mM, or about 0.86 mM to about 0.93 mM, or about 0.85 mM to about 0.94 mM, or about 0.83 mM to about 0.96 mM, or about 0.81 mM to about 0.98 mM, or about 0.8 mM, or about 0.75 mM, or about 0.7 mM or less, or about 1.0 mM, or about 1.05 mM, or about 1.1 mM or more.

In one embodiment, either the cell wash solution or the cell delivery solution can comprise any amount of sodium chloride to achieve the desired effect, including but not limited to about 119 mM to about 122 mM, or about 118 mM to about 123 mM, or about 117 mM to about 124 mM, or about 115 mM to about 126 mM, or about 113 mM to about 128 mM, or about 110 mM, or about 105 mM, or about 100 mM or less, or about 130 mM, or about 135 mM, or about 140 mM or more.

In another embodiment, either the cell wash solution or the cell delivery solution can comprise any amount of sodium chloride to achieve the desired effect, including but not limited to about 74 mM to about 77 mM, or about 73 mM to about 78 mM, or about 72 mM to about 79 mM, or about 70 mM to about 81 mM, or about 68 mM to about 83 mM, or about 66 mM to about 85 mM, or about 65 mM, or about 60 mM, or about 55 mM or less, or about 90 mM, or about 95 mM, or about 100 mM or more.

In another embodiment, either the cell wash solution or the cell delivery solution can comprise any amount of sodium chloride to achieve the desired effect, including but not limited to about 93 mM to about 96 mM, or about 92 mM to about 97 mM, or about 91 mM to about 98 mM, or about 90 mM to about 99 mM, or about 88 mM to about 101 mM, or about 85 mM to about 103 mM, or about 83 mM to about 105 mM, or about 80 mM, or about 75 mM, or about 70 mM or less, or about 105 mM, or about 110 mM or about 115 mM or more.

The one or more stabilizing agents of either the cell wash solution or the cell delivery solution can comprise any suitable stabilizing agent, such as one or more of a protein, such as one or more albumins, such as recombinant albumin (rHSA), Dextran (including Dextran 40, as one example), Poloxamer (including Poloxamer 188 as one example). Additionally, the one or more stabilizing agents can include one or more of the following, which also may be an excipient of either the cell wash solution or the cell delivery solution: polyethylene glycol, carboxymethyl cellulose, hyaluronic acid, starches, acrylates, methacrylates, polyvinyl alcohols, polyethylene oxides, polypropylene oxides, polyacrylates, polyvinylpyrrolidone, polymethacrylate, poly lactic-co-glycolic acids, polyacrylamides, polylactides, chitosans, gums, guar gums, xantham gums, carrageenans, hydroxypropyl cellulose, hydroxypropyl methyl cellulose, hydroxyethyl cellulose, cyclodextrin derivatives, beta-cyclodextrin derivatives, alginates, calcium alginates, and stearates.

In one embodiment, either the cell wash solution or the cell delivery solution can comprise any amount of a protein, such as one or more albumins, such as recombinant albumin (rHSA) to achieve the desired effect, including but not limited to about to about 0.07 w/w % to about 0.09 w/w %, or about 0.06 w/w % to about 0.1 w/w %, or about 0.05 w/w % to about 0.11 w/w %, or about 0.04 w/w % to about 0.12 w/w %, or about 0.03 w/w % to about 0.13 w/w %, or about 0.02 w/w % to about 0.15 w/w %, or about 0.01 w/w % to about 0.17 w/w %, or about 0.005 w/w % or less, or about 0.2 w/w %, or about 0.25 w/w % or more.

In another embodiment, either the cell wash solution or the cell delivery solution can comprise any amount of a protein, such as one or more albumins, such as recombinant albumin (rHSA) to achieve the desired effect, including but not limited to about 0.08 w/w % to about 0.11 w/w %, or about 0.07 w/w % to about 0.12 w/w %, or about 0.06 w/w % to about 0.13 w/w % or about 0.05 w/w % to about 0.14 w/w %, or about 0.04 w/w % to about 0.16 w/w %, or about 0.03 w/w % to about 0.18 w/w %, or about 0.02 w/w % to about 0.2 w/w %, or about 0.01 w/w % 10 about 0.22 w/w %, or about 0.05 w/w % or less, or about 0.25 w/w %, or about 0.3 w/w %, or about 0.35 w/w % or more.

In another embodiment, either the cell wash solution or the cell delivery solution can comprise any amount of a protein, such as one or more albumins, such as recombinant albumin (rHSA) to achieve the desired effect, including but not limited to about 6.50 w/w % to about 6.8 w/w %, or about 6.4 w/w % to about 6.9 w/w %, or about 6.3 w/w % to about 7.0 w/w %, or about 6.1 w/w % to about 7.2 w/w %, or about 5.9 w/w % to about 7.4 w/w %, or about 5.7 w/w % to about 7.6 w/w %, or about 5.5 w/w %, or about 5.0 w/w %, or about 4.5 w/w % or less, or about 8.0 w/w %, or about 8.5 w/w %, or about 9.0 w/w % or more.

In one embodiment, either the cell wash solution or the cell delivery solution can comprise any amount of Dextran to achieve the desired effect, including but not limited to about to 17.27 w/w % to about 17.30 w/w %, or about 17.26 w/w % to about 17.31 w/w %, or about 17.24 w/w % to about 17.33 w/w %, or about 17.2 w/w % to about 17.35 w/w %, or about 17.1 w/w % to about 17.4 w/w %, or about 17.0 w/w % to about 17.5 w/w %, or about 16.5 w/w %, or about 16.0 w/w %, or about 15.0 w/w % or less, or about 18.0 w/w %, or about 18.5 w/w %, or about 19.0 w/w % or more.

In one embodiment, either the cell wash solution or the cell delivery solution can comprise any amount of Dextran to achieve the desired effect, including but not limited to about to about 13.02 w/w % to about 13.05 w/w %, or about 13.0 w/w % to about 13.1 w/w %, or about 12.9 w/w % to about 13.2 w/w %, or about 12.7 w/w % to about 13.4 w/w %, or about 12.5 w/w % to about 13.5 w/w %, or about 12.2 w/w % to about 13.8 w/w %, or about 12.0 w/w %, or about 11.5 w/w %, or about 11.0 w/w % or less, or about 14.0 w/w %, or about 14.5 w/w %, or about 15.0 w/w % or more.

In one embodiment, either the cell wash solution or the cell delivery solution can comprise any amount of Poloxamer to achieve the desired effect, including but not limited to about to about 0.07 w/w % to about 0.09 w/w %, or about 0.06 w/w % to about 0.1 w/w %, or about 0.05 w/w % to about 0.11 w/w %, or about 0.04 w/w % to about 0.12 w/w %, or about 0.03 w/w % to about 0.13 w/w %, or about 0.02 w/w % to about 0.15 w/w %, or about 0.01 w/w % to about 0.17 w/w %, or about 0.005 w/w % or less, or about 0.2 w/w %, or about 0.25 w/w % or more.

There are several components that are not included in either the cell wash solution or the cell delivery solution and can be excluded wholly or below detectable limits. Some examples of components that can be excluded are: certain components of animal origin; certain stabilizing agents such as human serum albumin (HSA); certain salts such as zinc sulfate, sodium bicarbonate, and ferric nitrate; certain pH indicators such as Phenol Red; certain sources of energy such as sodium pyruvate; certain amino acids such as Glycine, L-Alanine, L-Arginine hydrochloride, L-Asparagine-H₂O, L-Glutamine, L-Cysteine, L-Histidine hydrochloride-H₂O, L-Isoleucine, L-Leucine, L-Lysine hydrochloride, L-Methionine, L-Phenylalanine, L-Proline, L-Serine, L-Threonine, L-Tryptophan, L-Tyrosine, and L-Valine; and certain vitamins such as Ascorbic Acid, Choline Chloride, D-Calcium pantothenate, Folic Acid, Niacinamide, Pyridoxal hydrochloride, Riboflavin, Thiamine hydrochloride, Vitamin B12 and i-Inositol.

The disclosure is also directed to methods of treating a subject. The method can comprise a step of administering a dose of a cell suspension for injection (CSI), with the CSI comprising a plurality of cells; one or more energy source components; one or more pH buffers; one or more salts; and one or more stabilizing agents. The plurality of cells can include any of the cells disclosed herein and can be cells of the subject undergoing the treatment, or cells from another source or subject. The subjects treated with the methods can be adults or pediatric. The subjects treated with the methods of the disclosure can be mammalian and are preferably human. The subjects may also be animals such as primates, canines, felines, livestock or horses.

The number of cells being administered can vary based on the specific treatment. The number of cells can be any suitable and efficacious number, including but not limited to about 10 cells or less, about 100 cells, about 1,000 cells, about 10,000 cells, about 100,000 cells, about 10⁶ cells, about 10⁷ cells, about 10⁸ cells, about 10⁹ cells, about 10¹⁰ cells, about 10¹¹ cells, about 10¹⁵ cells, about 10²⁰ cells, or more, including any ranges with any of these values as endpoints. These number of cells can be administered in any suitable volume, at any suitable concentration of cells, such as about 25×10⁶ cells/mL to about 150×10⁶ cells/mL, or about 50×10⁶ cells/mL to about 100×10⁶ cells/mL, or about 60×10⁶ cells/mL to about 90×10⁶ cells/mL, or about 65×10⁶ cells/mL to about 85×10⁶ cells/mL, or about 70×10⁶ cells/mL to about 80×10⁶ cells/mL, or about 50×10⁶ cells/mL, or about 75×10⁶ cells/mL, or about 100×10⁶ cells/mL, or about 125×10⁶ cells/mL, or about 150×10⁶ cells/mL. In one embodiment, the number of cells administered can be 120×10⁶ cells/mL, 130×10⁶ cells/mL or 140×10⁶ cells/mL.

The method can be directed to a single administration of one dose of the CSI, or the method can include the further steps of waiting a predetermined time after the previous administration and then administering another dose of the CSI, which can be the same dose kind of dose with the same or similar components, or a different dose of CSI. The method can be directed to unilateral or bilateral administration, e.g. injection such as ICV injection. The predetermined amount of time can vary based on the treatment and can be any suitable amount of time, such as about 1 minute, about 1 hour, about 3 hours, about 6 hours, about 12 hours, about 24 hours, about 2 days, about 4 days, about 7 days, about 10 days, about 14 days, about 21 days, about 1 month, about 6 weeks, about 2 months, about 4 months, about 6 months, about 9 months, about 12 months, about 18 months, about 2 years or more, including any ranges with any of these values as endpoints.

These additional steps of waiting a predetermined time after the previous administration and then administering another dose of the CSI can repeat based on the treatment and can be any suitable amounts of repetitions, such as two, three, four, five, six, seven, eight, nine, ten, or more times.

The method of treatment can also include a prior step to the administration of the CSI, or a concurrent step to the administration of the CSI, or a subsequent step to the administration of the CSI. This optional concurrent step or subsequent step can be one or more of administering a treatment, the treatment selected from the group consisting of an immunosuppressant agent, a chemotherapeutic agent, a radiation treatment, a bone marrow treatment, a hormone treatment, a surgical treatment or any treatments aiming at improving the subject response to the administration of the CSI. Each of these treatments can be varied based on the treatment needed.

The disclosure is also directed to methods of administering a dosage volume to a subject. This method of administering the dosage volume includes loading a dose delivery device, such as a syringe or any other device able to deliver a solution to a subject, with the dosage volume of a cell suspension for injection (CSI). The CSI can include a plurality of cells; one or more energy source components; one or more pH buffers; one or more salts; and one or more stabilizing agents, wherein the one or more stabilizing agents are selected from the group consisting of recombinant albumin (rHSA), Dextran, and Poloxamer. The plurality of cells can be differentiated midbrain dopaminergic neuronal cells and can comprise a range of about 10° cells to about 10¹⁰ cells, or a range of about 10⁸ cells to about 10¹⁰ cells.

After loading the solution delivery device with the dosage volume, the method then includes a step of injecting the dosage volume into a portion of the subject. The subject can be a human, and this injecting step can occur directly into a portion of the subject, and/or into an intravenous line that is in a blood vessel of the subject.

This method of administration can further comprise a concurrent step or a subsequent step of administering a treatment, the treatment selected from the group consisting of a chemotherapeutic agent, a radiation treatment, a bone marrow treatment, a hormone treatment, and a surgical treatment.

The methods and model of the present disclosure will be better understood by reference to the following Examples, which are provided as exemplary of the disclosure and not in any way limiting.

### Example 1

An example of forming a cell wash solution of the disclosure is disclosed in this example.

This cell wash solution can be used to wash any cells, including cells in a cryoprotectant, after being thawed. The procedure is described for a 1 kg scale but can be scaled up or down accordingly.

| Material | Chemical Formula/Acronym |
|---|---|
| HEPES | C₈H₁₈N₂O₄S |
| Calcium Chloride, dihydrate | CaCl₂•2H₂O |
| Magnesium Chloride, hexahydrate | MgCl₂•6H₂O |
| Potassium Chloride, anhydrous | KCl |
| Sodium Chloride, anhydrous | NaCl |
| Sodium Phosphate, monobasic, dihydrate | NaH₂PO₄•2H₂O |
| Dextrose (D-glucose), monohydrate | C₆H₁₂O₆•H₂O |
| Recombinant Albumin | rHSA |
| Water for Injection (WFI) | H₂O |

With these starting materials, the following procedure was followed:
1. Tare a 1 L container with a stir bar and fill with 500 g of water for injection (WFI) using serological pipette.
2. Using a balance, tare separate weight boats and weigh out each reagent. Quantitatively transfer to the 1 L bottle while stirring on a magnetic stir plate.
3. Complete weight to 980.0 g with WFI, considering volume of water already added in the previous steps.
4. Adjust bulk solution pH to about 6.95-about 7.05 at about 17° C.-about 25° C. with 1 M NaOH and 1 M HCl solutions. Record volume of additions (approx. 3.500 mL of 1M NaOH).
5. Add rHSA and up to 20.0 mL of WFI (to bring total weight to 1 kg), considering the volume of acid and base used to adjust the pH of the solution in step 3. Place on magnetic stir plate until well mixed, about 20-about 30 minutes.
6. Take an aliquot to measure osmolality.
   a. This in-process measurement is expected to be about 295 mOsm/kg H₂O.
7. Using a sterile filtration apparatus in a sterile environment, filter solution.
8. Take an aliquot in a sterile environment to confirm for final formulation pH, osmolality, and density are sufficient.
   a. pH should be between about 6.9-about 7.1 at about 17-about 25° C., about 7.2-about 7.4 at about 0-about 5° C.
   b. Osmolality should be between about 282-about 302 mOsm/kg H₂O.¹ ¹Osmolality specification is based on a conversion (according to USP <785>) from the osmolarity specification of 280-300 mOsm/L, given the density is about 1.0090 g/mL and solute concentration is about 0.0167617 g/mL.
   c. Density should be about 1.0075-about 1.0105 g/mL.
9. Store at about 4° C. (or as desired/needed).

### Example 2

An example of forming a cell delivery solution of the disclosure is provided in this example.

This solution can be used to resuspend cells during preparation of a cell suspension for injection (CSI). The procedure is described for a 1 kg scale but can be scaled up or down accordingly.

| Material | Chemical Formula/Acronym |
|---|---|
| HEPES | C₈H₁₈N₂O₄S |
| Calcium Chloride, dihydrate | CaCl₂•2H₂O |
| Magnesium Chloride, hexahydrate | MgCl₂•6H₂O |
| Potassium Chloride, anhydrous | KCl |
| Sodium Chloride, anhydrous | NaCl |
| Sodium Phosphate, monobasic, dihydrate | NaH₂PO₄•2H₂O |
| Dextrose (D-glucose), monohydrate | C₆H₁₂O₆•H₂O |
| Recombinant Albumin | rHSA₍ₛ₎ |
| Dextran 40 | Dex40 |
| Poloxamer P188 | P188 |
| Water for Injection (WFI) | H₂O |

With these starting materials, the following procedure was followed:
1. Fill a 1 L container with 500 g of water for injection (WFI).
2. Using an appropriate balance, tare separate weight boats and weigh out each reagent. Quantitatively transfer to the 1 L bottle while stirring on a magnetic stir plate.
3. Complete mass to about 750.0 g with WFI, considering amount of water already added in the previous step. Allow to solubilize for about 30-about 45 minutes.
4. Adjust bulk solution pH to about 7.00-about 7.05 at about 17-about 25° C. with 1 M NaOH and 1 M HCl solutions. Record volume of additions (approx. 3.000-3.125 mL of NaOH).
5. Add rHSA(s) under constant stirring. Allow to dissolve for about 30-about 45 minute.
6. Add Dextran 40 under constant stirring. Allow to dissolve for about 30-about 45 minutes at medium speed.
7. Top up bulk with WFI to 1 kg, considering the volume of acid and base used to adjust the pH of the solution in step 4. Place on magnetic stir plate until well mixed, about 5-about 10 minutes.
8. Take an aliquot to measure osmolality.
   This in-process measurement is expected to be about 344 mOsm/kg H₂O.
9. Using a sterile filtration apparatus in a sterile environment, filter solution.
10. Take an aliquot in a sterile environment to confirm for final formulation pH, osmolality, and density are sufficient.
   pH should be between about 6.9-about 7.1 at about 17-about 25° C.
   a. Osmolality should be between about 341-about 366 mOsm/kg H₂O.
      This would correspond to an osmolarity of about 280-about 300 mOsm/L based on a density of about 1.081 g/mL at about 5° C. and a solute concentration of about 0.249 g/mL.
   b. Density should be about 1.0800-about 1.0820 g/mL at about 5° C. However, the final density of the CSI can include one or more additional components, including a substantially small or a substantially trace amount of cell wash solution and/or a buffer. The presence of these one or more additional components can have substantially no affect on density of the cell delivery solution, a substantially small affect on density of the cell delivery solution (e.g. about 1% change, about 2% change, about 3% change, about 4% change, about 5% change, or greater) or a substantially large affect on density of the cell delivery solution (e.g. about 10% change, about 11% change, about 12% change, about 13% change, about 14% change, about 15% change, or greater).
11. Store at 2-8° C. (or as desired/needed).

### Example 3

The following example of the present disclosure is intended to be illustrative of an embodiment of the cell wash solution and the cell delivery solution when used with microglia cells.

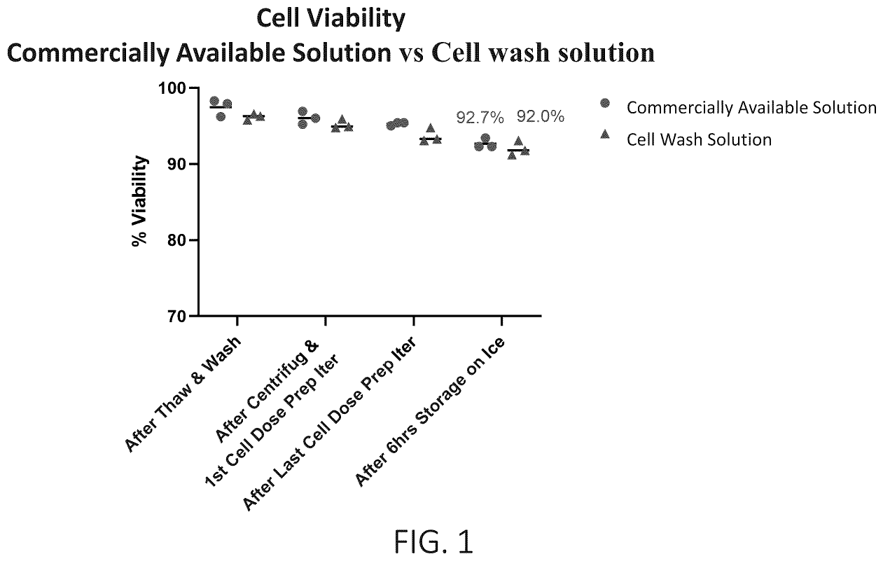

All data presented in FIGS. 1-4 are directed to microglia cells that had been cryopreserved. After thawing the frozen cells, the cryoprotectant was washed from the cells by the addition of an embodiment of the cell wash solution. The mixture containing cells was then centrifuged to pellet the cells, so the cell wash solution supernatant could be removed. After the supernatant was removed, the cells were resuspended in an embodiment of the cell delivery solution, forming the cell suspension for injection (CSI).

The CSI was then stored for 0, 8, or 24 h and characterized by several assays. The control group used was a commercially available solution that contained a high number of excipients or some excipients that must be added prior to use, making this solution not ideal with late stage clinical and commercial use.

As can be seen in FIG. 1, the cell viability of cells was evaluated at different points in a typical preparation of cells for injection. After six hours of storage in a container on ice, the microglia cells stored in a cell wash solution of the present disclosure had nearly the same cell viability (92.0%) as compared to the cell viability of cells the commercially available solution (92.7%).

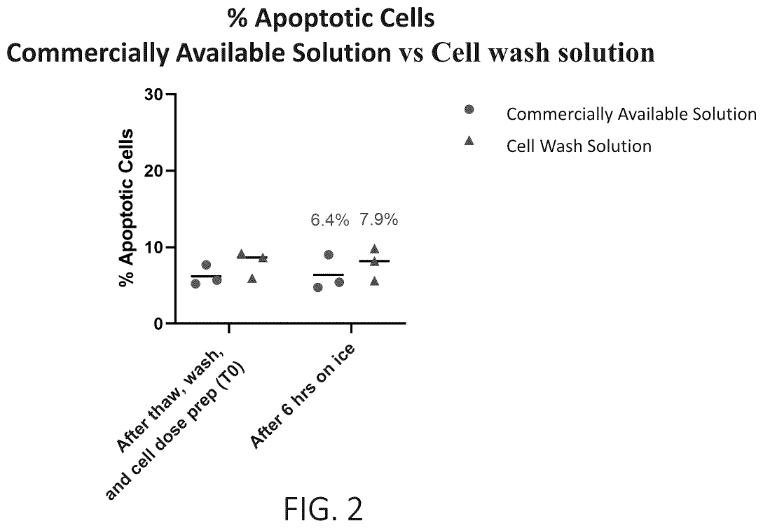

Apoptosis is a process in which a cell enters a state of cell death. As can be seen in FIG. 2, the percentage of apoptotic cells in the cells stored in the commercially available solution as compared to the cells stored in a cell wash solution of the present disclosure had nearly the same percentage of apoptotic cells (˜7.9%) as compared to the percentage of apoptotic cells in the commercially available solution (˜6.4%) after six hours of storage in a container on ice.

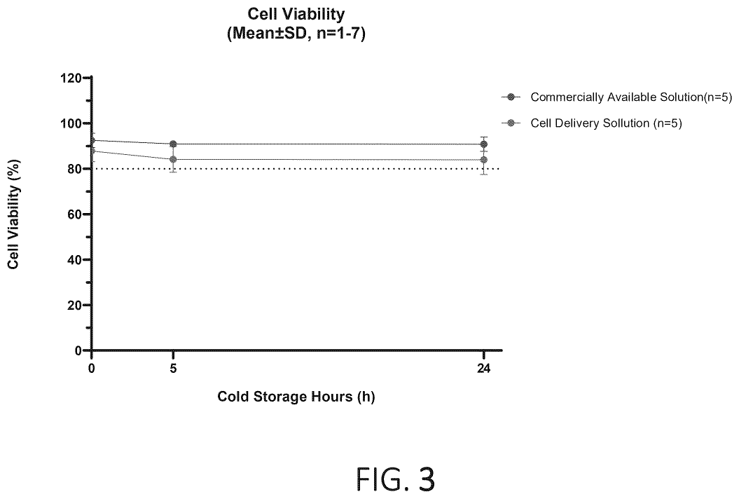

As can be seen in FIG. 3, after 24 hours of storage in a container on ice, the microglia cells stored in a cell delivery solution of the present disclosure had nearly the same cell viability (around 90%) as compared to the cell viability of cells the commercially available solution (around 80%).

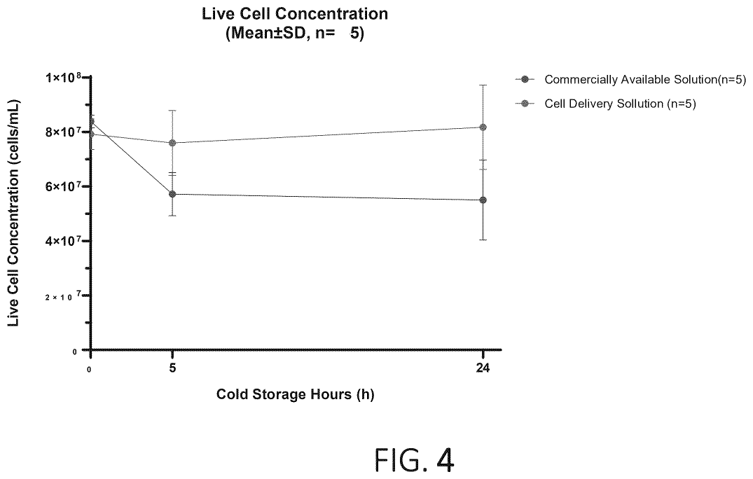

As can be seen in FIG. 4, the microglia cell dose (evaluated by live cell concentration) was substantially stable after 24 hours of storage, when the cells were stored in a cell delivery solution of the present disclosure, and not significantly different from the stability of cells stored in a commercially available solution.

### Example 4

The following example of the present disclosure is intended to be illustrative of an embodiment of the cell wash solution and the cell delivery solution when used with dopamine producing cells.

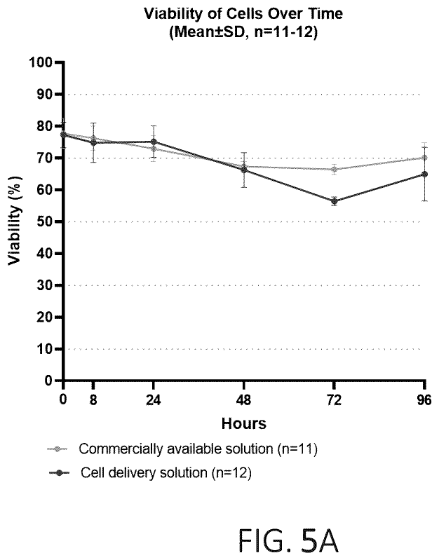

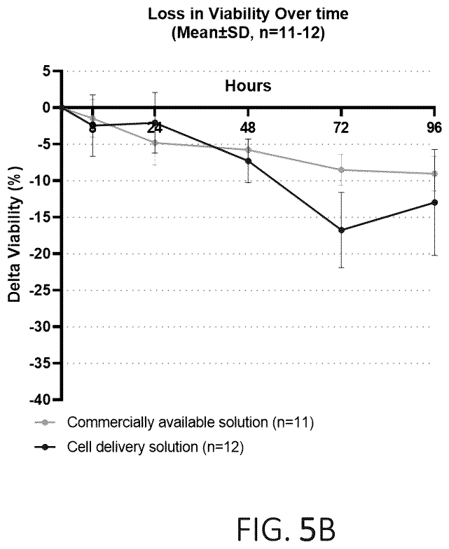

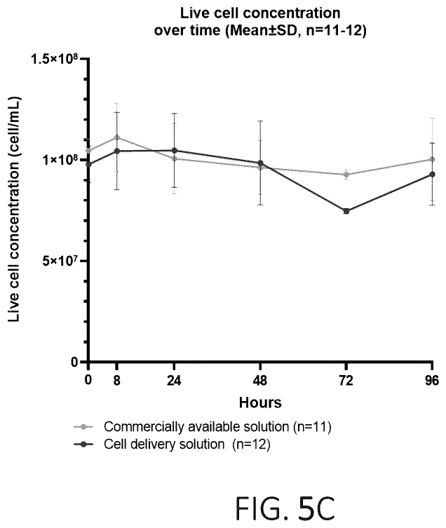

All data presented in FIGS. 5-12 are directed to dopamine-producing cells that had been cryopreserved. After thawing the frozen cells, the cryoprotectant was washed from the cells by the addition of an embodiment of the cell wash solution. The solution containing cells was then centrifuged to remove the supernatant and the cells were resuspended in an embodiment cell delivery solution, forming the cell suspension for injection (CSI). The CSI was then stored for 0, 8, 24 or 48 h. The control used was a commercially available solution that contains non-components of human origin.

As can be seen in FIG. 5A-C, the cell viability and cell dose are substantially stable after 96 hours of storage. The gray lines and data points in FIGS. 5A-5C represent the average of eleven samples of cells stored in a commercially available solution. The blue lines and data points in FIGS. 5A-5C represent the average of twelve samples of cells stored in an embodiment of the cell delivery solution.

As seen in FIG. 5B, there was minimal loss in viability of cells stored for over 24 hours in cell delivery solutions (˜2.5%) which was lower than the loss in viability of cells stored in the commercially available solution (˜5%).

As seen in FIG. 5C, in all formulations tested, live cell dose stayed within the clinical target dose of about 100E6±10 cell/mL, up to 96 hours.

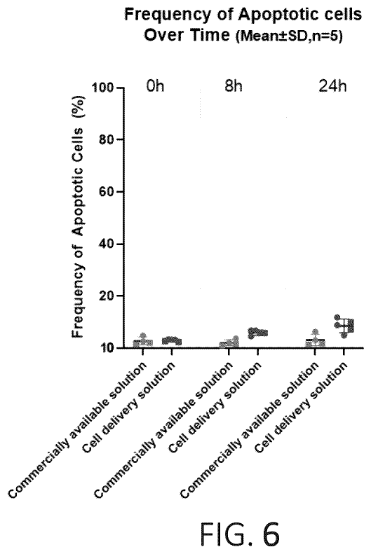

Apoptosis is the process that describes early cell death. Further data of apoptotic cells is shown in FIG. 6. The gray data points in FIG. 6 represent the average of five samples of cells stored in a commercially available solution. The blue data points in FIG. 6 represent the average of five samples of cells stored in an embodiment of the cell delivery solution. Both the cells suspended in the commercially available solution and the cell delivery solution had low apoptosis level (<10%) after 24h in storage.

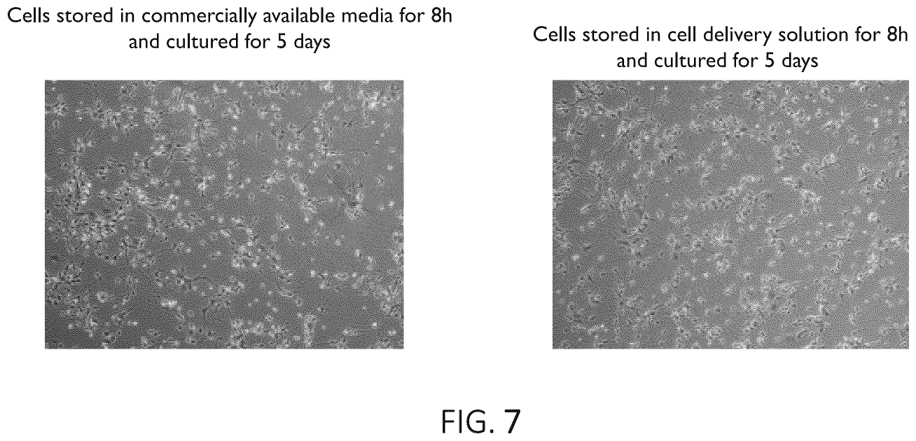

The influence of the formulation on dopamine-producing cell morphology after storage was assessed when cells were formulated in a commercially available solution or the embodiment of the cell delivery solution. FIG. 7 shows representative pictures of cells cultured after storage in commercially available solution (left) or in the cell delivery solution (right). As can be seen in FIG. 5, no visual differences were observed in cell morphology, and morphology was as expected after the 5 days of culturing.

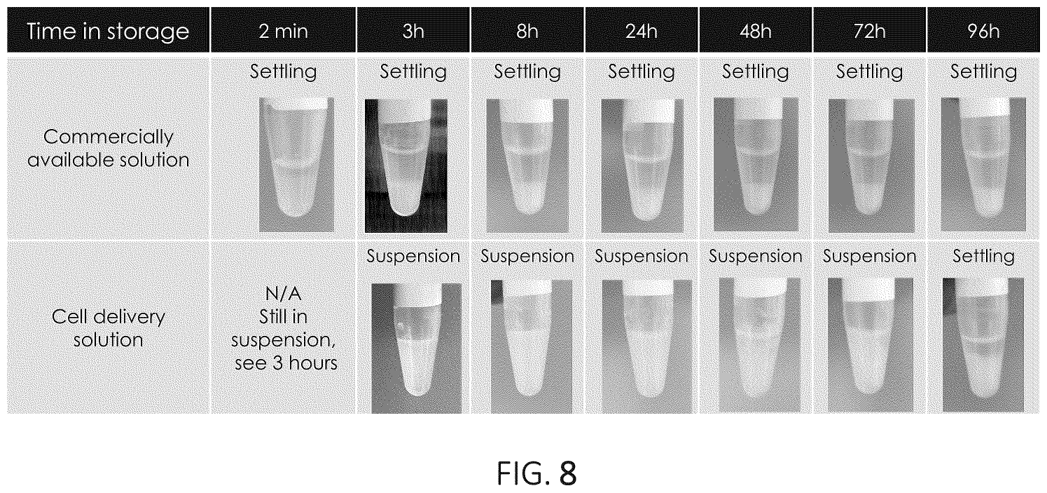

An advantage of an embodiment of the cell delivery solution is the ability to maintain cells in suspension to improve dose assurance. In this example, cells were suspended in a cell delivery solution or in a commercially available solution. As can be seen in FIG. 8, and demonstrated by serial photographs of containers over time, an embodiment of the cell delivery solution maintained cells in suspension at least 72 hours, which is in contrast to the commercially available solution, in which cells settle within minutes. As can be seen in FIG. 8, visual non-homogeneity of cells in the embodiment of the cell delivery solution begins around 96 hours, which is significantly longer than cells in the commercially available solution.

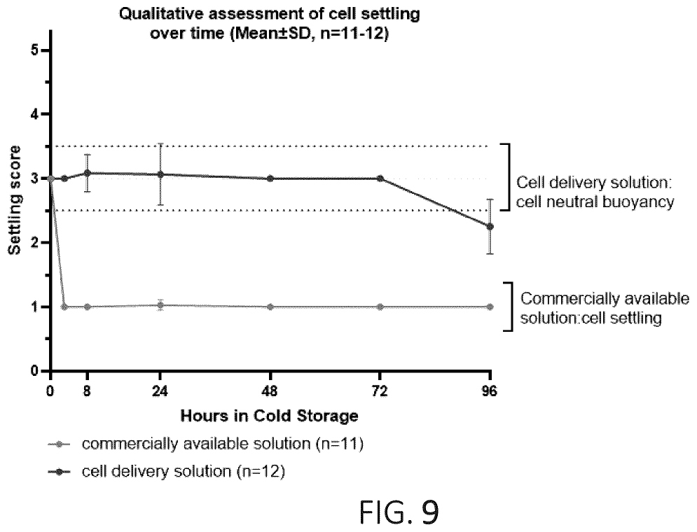

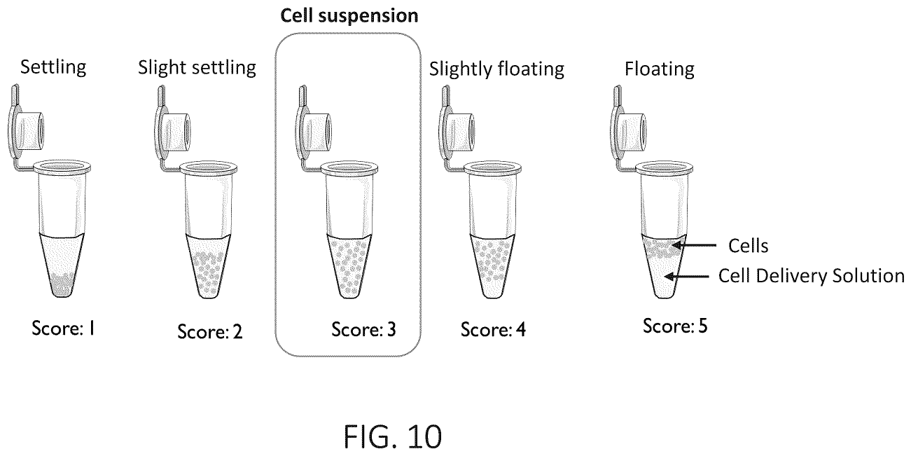

FIG. 9 is a graphical illustration of the results visualized from FIG. 8. At each time point, the container was visually observed and scored according to the scoring system shown in FIG. 10, with a score of 3 being the initial score upon mixing, when cells are homogenously distributed in the solution. As can be seen in the results in FIG. 9, graphed over time, the cells resuspended in the commercially available solution are quickly subject to settling to a score of 1. As can also be seen in the results, the embodiment of the cell delivery solution maintained cells in suspension with a score of 3 for a significant amount of time (72h).

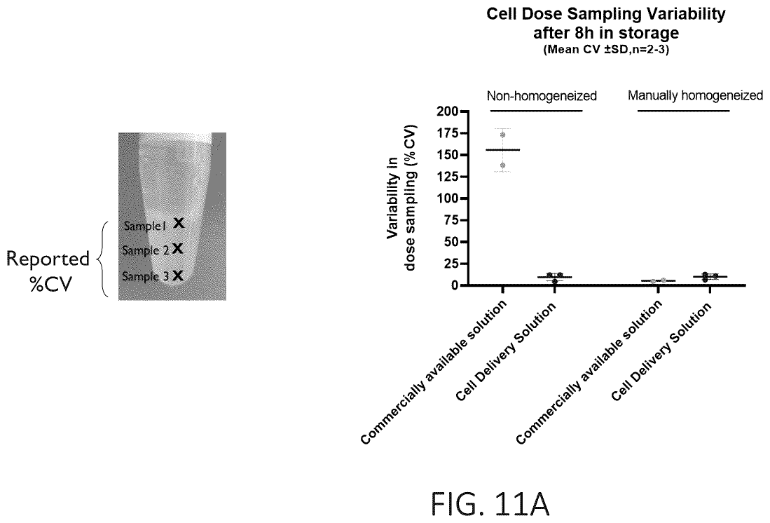

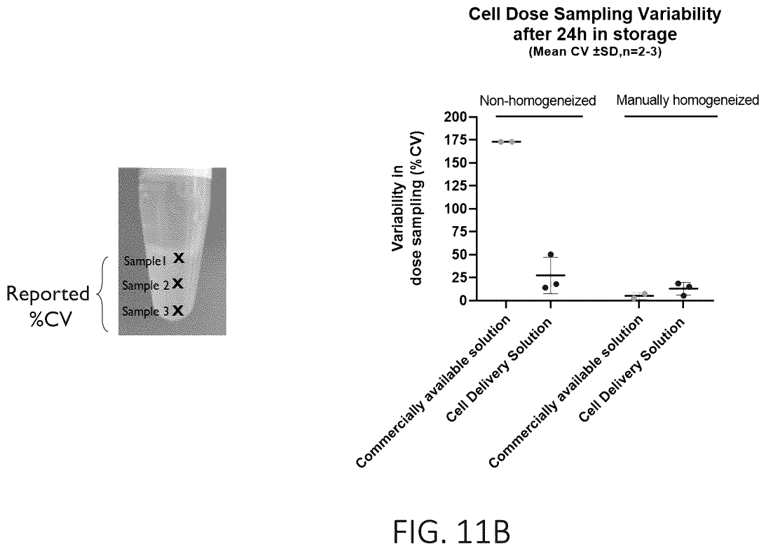

FIGS. 11A-11B are graphical representations of a quantitative assessment of cell settling. The cell suspension is sampled three times: in the top, middle and lower part of the container, and the variability between the three measurements is calculated as the coefficient of variation (% CV). In this scenario, the greater the % CV, the more the cells have settled.

The gray data points in FIGS. 11A-11B represent variability of dose sampling for cells stored in the commercially available solution. The blue points in FIGS. 11A-11B represent variability of dose sampling for cells stored in two embodiments of the cell delivery solution: “A” and “B”. Specifically, FIGS. 11A-11B demonstrate that two different cell delivery solutions, A and B, reduce cell settling up to 24 hours, compared to the commercially available solution.

A comparison can be made amongst FIG. 11A, which demonstrates variability in dose sampling after 8 hours in storage, to FIG. 11B, which demonstrates variability in dose sampling after 24 hours in storage. This comparison indicates that cell delivery solutions A and B maintain cells in suspension at least 24h (% CV<25%), which is significantly better than cells stored in the commercially available solution, in which the cell suspension is non-homogenous (% CV˜150-175) after at most 8 hours of storage.

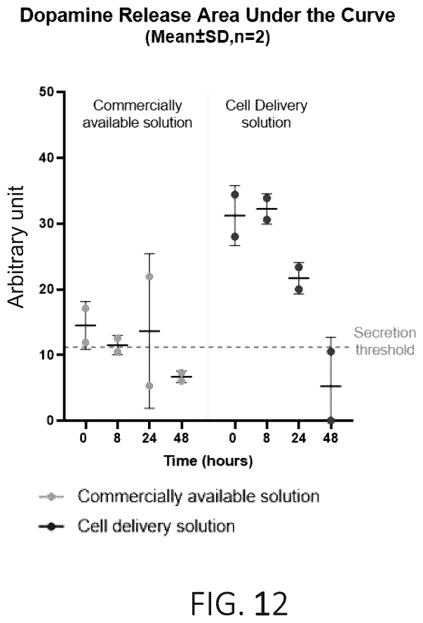

As can be seen in FIG. 12, dopamine producing cells were able to produce dopamine above a certain threshold, whether the cells have been in storage in a commercially available solution or in an embodiment of the cell delivery solution for 0, 8 or 24h.

Overall, the data presented here suggest that dopamine-producing cells stay healthy and functional whether they have been stored in a commercially available solution or in an embodiment of the cell delivery solution. Advantages of using the cell delivery solution include: the superior dose assurance since cells stay in suspension for extended period of time; a greater manufacturing and commercial relevance since the formulation contains a minimal number of pharmaceutical grade components and no components of human or animal origin; and the ease of use since the formulation can be stored in standard storage condition and is ready to use.

### Example 5

In this example a cell wash solution (CWS) of the disclosure was evaluated as a component of a cell solution for injection (CSI). In this example the CWS containing CSI, and a commercially available solution, were evaluated for delivery of two different concentrations of cells, ˜75,000 cells/μL and ˜20,000 cells/μL. The tests were done in triplicate and the data presented in this example is the average of these triplicate data.

Each of the CSI of this example and the commercially available solution were each prepared with both a cell concertation of ˜75,000 cells/μL and a cell concentration of ˜20,000 cells/μL, with cell viability/live cell concentration and apoptosis of the cells measured at about 0 hours and at about 6 hours of storage at 2° C.-8° C.

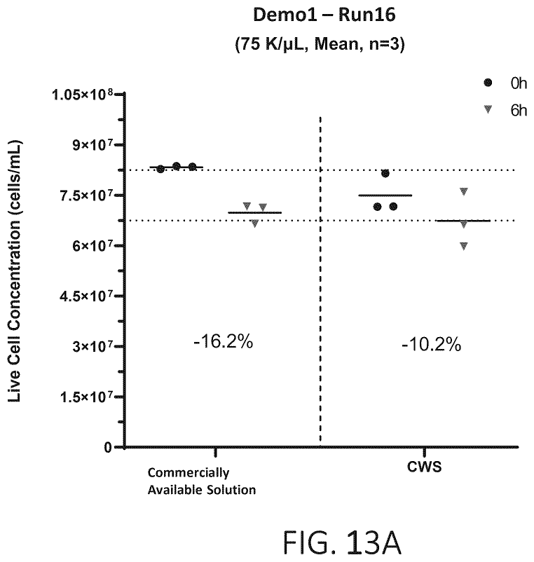

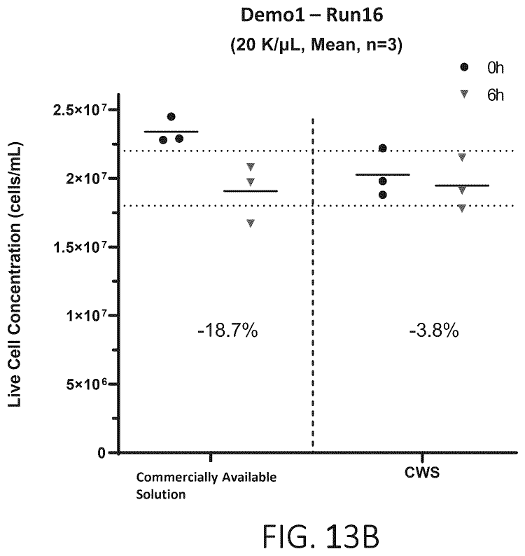

FIG. 13A illustrates the results at ˜0 hours and ˜6 hours for ˜75,000 cells/μL stored in both a commercially available solution, and in an embodiment of CWS. FIG. 13B illustrates the results at ˜0 hours and ˜6 hours for ˜20,000 cells/μL stored in both a commercially available solution, and in an embodiment of CWS. As can be seen from FIGS. 13A and 13B, at both cell concentrations, the CWS of the present disclosure had a smaller decrease in live cell concentration as compared to the commercially available solution.

Specifically, in FIG. 13A, at a cell concentration of ˜75,000 cells/μL, after about 6 hours of storage time, there was about a 10.2% reduction in the live cell concentration for cells stored in the CWS of the present disclosure as compared to about a 16.2% reduction in live cell concentration for cells stored in the commercially available solution. In FIG. 13B, at a cell concentration of ˜20,000 cells/μL, after about 6 hours of storage time, there was about a 3.8% reduction in the live cell concentration for cells stored in the CWS of the present disclosure as compared to about a 18.7% reduction in live cell concentration for cells stored in the commercially available solution.

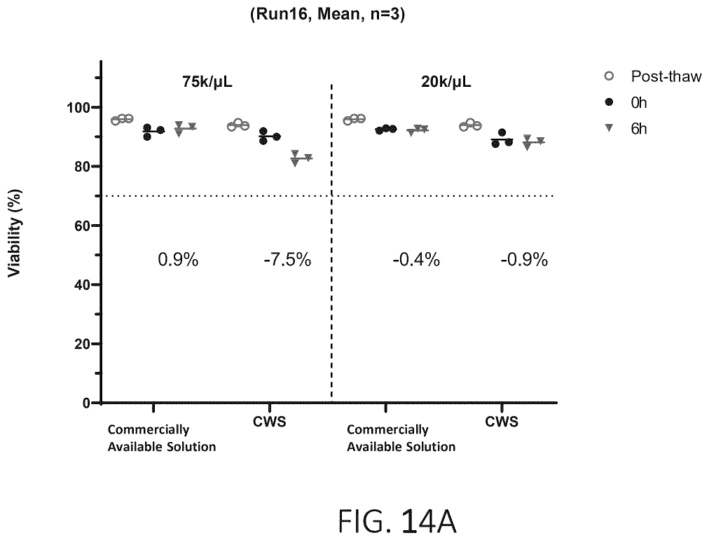

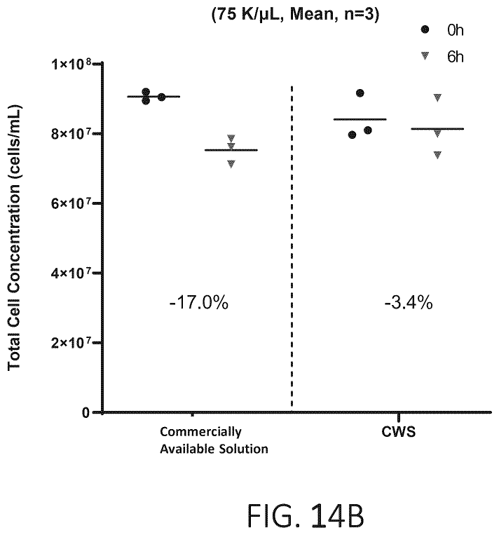

In the same experiment, cell viability of cells stored in the CWS of the present disclosure as compared to the commercially available solution was determined, as shown in FIGS. 14A and 14B.

FIG. 14A illustrates the results at post-thaw of the cells, and storage of the cells for ˜0 hours and ˜6 hours for both ˜75.000 cells/μL and ˜20,000 cells/μL, stored in both a commercially available solution, and in an embodiment of CWS. As can be seen from FIG. 14A, at both cell concentrations, the CWS of the present disclosure has a comparable cell viability percentage to the commercially available solution after about 6 hours, and maintains cell viability well over 70% after about 6 hours storage.

FIG. 14B is an illustration of total cell concentration for cells of the initial concentration of ˜75,000 cells/μL at ˜0 hours and ˜6 hours stored in both a commercially available solution, and in an embodiment of CWS. As can be seen from FIG. 14B, the CWS of the present disclosure has a smaller decrease in total cell concentration (˜3.4%) as compared to cells stored in the commercially available solution (˜17%).

A measure of morphology was also for conducted for cells stored for ˜0 hours and ˜6 hours, at 2° C.-8° C., for both ˜75.000 cells/μL and ˜25,000 cells/μL, stored in both a commercially available solution, and in an embodiment of CWS. This test was done in triplicate, with ˜100,000 cells plated per 96 well plate.

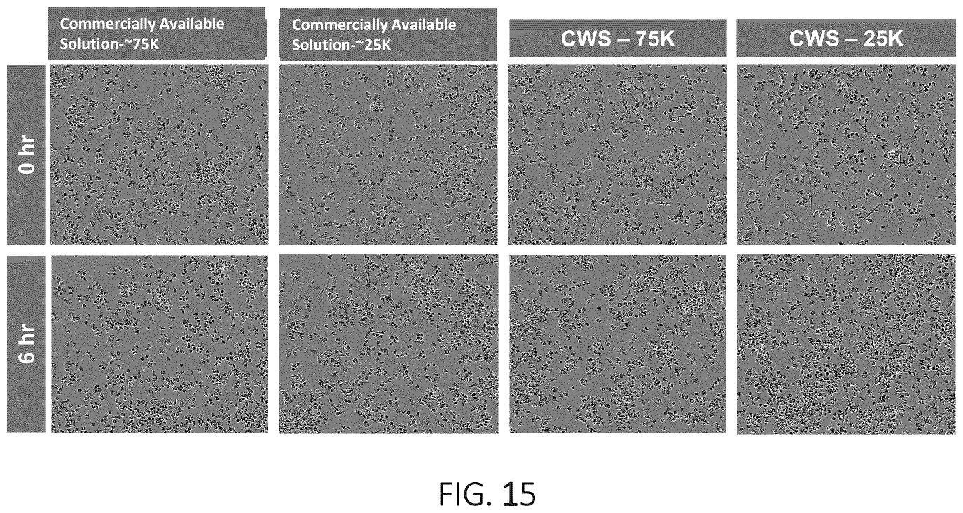

Representative images were taken, the results are shown in the series of photographs of FIG. 15. As shown in FIG. 15, no visual differences were observed in cell morphology, and morphology was as expected after ˜6 hours of storage in both the CWS of the present disclosure and the commercially available solution, at both cell concentrations.

The described embodiments and examples of the present disclosure are intended to be illustrative rather than restrictive and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the compositions illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A cell delivery solution, the solution comprising:
one or more energy source components;
one or more pH buffers;
one or more salts; and
one or more stabilizing agents, wherein the one or more stabilizing agents are selected from the group consisting of recombinant albumin (rHSA), Dextran, and Poloxamer.

2. The solution of claim 1, wherein the one or more energy source components comprise a sugar.

3. The solution of claim 2, wherein the sugar is dextrose.

4. The solution of claim 1, wherein the one or more pH buffers comprise one or more zwitterionic organic chemical buffering agents.

5. The solution of claim 4, wherein the one or more zwitterionic organic chemical buffering agents comprise 4-(2-hydroxyethyl)-1-piperazineethanesulfonic acid (HEPES).

6. The solution of claim 1, wherein the one or more salts are selected from the group consisting of calcium chloride, magnesium chloride, potassium chloride, sodium phosphate monobasic and sodium chloride.

7. The solution of claim 1, wherein the solution does not include human serum albumin (HSA).

8. The solution of claim 1, wherein the solution does not include any components of animal origin.

9. The solution of claim 1, wherein the solution does not include one or more of zinc sulfate, sodium bicarbonate, and ferric nitrate.

10. The solution of claim 1, wherein the solution does not include a pH indicator.

11. The solution of claim 10, wherein the pH indicator is Phenol Red.

12. The solution of claim 1, wherein the solution does not include sodium pyruvate.

13. The solution of claim 1, wherein the solution does not include an amino acid.

14. The solution of claim 1, wherein the solution does not include a vitamin.

15. The solution of claim 1, further comprising one or more excipients selected from the group consisting of polyethylene glycol, carboxymethyl cellulose, hyaluronic acid, starches, acrylates, methacrylates, polyvinyl alcohols, polyethylene oxides, polypropylene oxides, polyacrylates, polyvinylpyrrolidone, polymethacrylate, poly lactic-co-glycolic acids, polyacrylamides, polylactides, chitosans, gums, guar gums, xantham gums, carrageenans, hydroxypropyl cellulose, hydroxypropyl methyl cellulose, hydroxyethyl cellulose, cyclodextrin derivatives, beta-cyclodextrin derivatives, alginates, calcium alginates, and stearates.

16. A cell suspension for injection, the suspension comprising:
a plurality of cells;
one or more energy source components;
one or more pH buffers;
one or more salts; and
one or more stabilizing agents, wherein the one or more stabilizing agents are selected from the group consisting of recombinant albumin (rHSA), Dextran, and Poloxamer.

17. The suspension of claim 16, wherein the cells are differentiated midbrain dopaminergic neuronal cells.

18. The suspension of claim 17, wherein the differentiated midbrain dopaminergic neuronal cells are differentiated midbrain dopaminergic neuron progenitor cells.

19. The suspension of claim 16, further comprising one or both of a cell wash buffer and a cryoprotectant.

20. The suspension of claim 19, wherein the cell wash buffer is selected from the group consisting of a phosphate buffer saline (PBS), 4-(2-hydroxyethyl)-1-piperazineethanesulfonic acid (HEPES), Bio-Plex Pro™ Cell Signaling Wash Buffer, and Cultrex™ 3-D Cell Wash Buffer.

21. The suspension of claim 19, wherein the cryoprotectant is selected from the group consisting of a permeating-type cryoprotectant and a non-permeating-type cryoprotectant.

22. The suspension of claim 21, wherein the permeating-type cryoprotectant is selected from the group consisting of dimethyl sulfoxide (DMSO), glycerol, propylene glycol, and ethylene glycol.

23. The suspension of claim 21, wherein the non-permeating-type cryoprotectant is selected from the group consisting of sucrose, carboxymethylcellulose salts, carboxymethylcellulose (CMC), monosaccharide, and disaccharide.

24. The suspension of claim 16, wherein the one or more energy source components comprise a sugar.

25. The suspension of claim 24, wherein the sugar is dextrose.

26. The suspension of claim 16, wherein the one or more pH buffers comprise one or more zwitterionic organic chemical buffering agents.

27. The suspension of claim 26, wherein the one or more zwitterionic organic chemical buffering agents comprise 4-(2-hydroxyethyl)-1-piperazineethanesulfonic acid (HEPES).

28. The suspension of claim 16, wherein the one or more salts are selected from the group consisting of calcium chloride, magnesium chloride, potassium chloride, sodium phosphate monobasic and sodium chloride.

29. The suspension of claim 16, wherein the suspension does not include human serum albumin (HSA).

30. The suspension of claim 16, wherein the suspension does not include any components of animal origin.

31. The suspension of claim 16, wherein the suspension does not include one or more of zinc sulfate, sodium bicarbonate, and ferric nitrate.

32. The suspension of claim 16, wherein the solution does not include a pH indicator.

33. The suspension of claim 32, wherein the pH indicator is Phenol Red.

34. The suspension of claim 16, wherein the suspension does not include sodium pyruvate.

35. The suspension of claim 16, wherein the suspension does not include an amino acid.

36. The suspension of claim 16, wherein the suspension does not include a vitamin.

37.-91. (canceled)
